(19) United States
(12) Patent Application Publication
(10) Pub. No.: US 2013/0282005 A1
(43) Pub. Date: Oct. 24, 2013

(54) CATHETER NAVIGATION SYSTEM

(71) Applicants: Martin Koch, Nurnberg (DE); Atilla Peter Kiraly, Plainsboro, NJ (US); Norbert Strobel, Heroldsbach (DE); Alexander Benjamin Brost, Erlangen (DE)

(72) Inventors: Martin Koch, Nurnberg (DE); Atilla Peter Kiraly, Plainsboro, NJ (US); Norbert Strobel, Heroldsbach (DE); Alexander Benjamin Brost, Erlangen (DE)

(73) Assignees: SIEMENS CORPORATION, Iselin, NJ (US); SIEMENS AKTIENGESELLSCHAFT, Munchen (DE); Friedrich-Alexander-Universitat Erlangen-Numberg, Erlangen (DE)

(21) Appl. No.: 13/859,115

(22) Filed: Apr. 9, 2013

Related U.S. Application Data
(60) Provisional application No. 61/637,478, filed on Apr. 24, 2012.

(51) Int. Cl. A61B 18/14

(52) U.S. Cl. CPC A61B 18/1492
USPC 606/41

(57) ABSTRACT

An integrated catheter navigation system (100) and method (200) that combines anatomical imaging (302, 304) with catheter tip-to force information (306, 308) during an ablation procedure.

### CROSS-REFERENCE TO RELATED APPLICATIONS

This application claims the benefit of Provisional U.S. Patent Application Ser. No. 61/637,478 entitled, “Navigation System with Contact Force Assessment”, filed in the name of Martin Willibald Koch, Atilla Peter Kiraly, Norbert Strobel and Alexander Benjamin Brost, on Apr. 24, 2012, the disclosure of which is also hereby incorporated herein by reference.

### FIELD OF INVENTION

The present invention relates to catheter tracking and navigation. More particularly, the present invention relates to a catheter navigation system especially useful in the ablation treatment of heart arrhythmias.

### BACKGROUND OF THE INVENTION

Heart arrhythmia are usually caused by improper or abnormal coordination of electrical impulses in a patient's heart. They can present themselves as a fast, slow, or irregular heart beat. Atrial fibrillation (Afib) is a form of abnormal heart rhythm. One type of atrial fibrillation is paroxysmal atrial fibrillation in which the heart has an irregular heartbeat occurring every so often that then returns to its normal rhythm. The causes of paroxysmal atrial fibrillation are unknown and episodes are hard to predict. Consequently, paroxysmal atrial fibrillation is typically treated in the first instance with pharmacotherapy. However, once pharmacotherapy fails, the common option for treatment is the electrical isolation of the pulmonary veins from the left atrium via catheter ablation.

Electrophysiology (EP) procedures or studies, which include catheter ablation, are conducted by cardiac medical specialists to help diagnose and treat abnormal heart rhythms of patients. This is generally described in an article by M. Haissaguerre, L. Gencel, B. Fischer, P. Le Metayer, F. Poquet, F. I. Marcus, and J. Clementy, entitled “Successful Catheter Ablation of Atrial Fibrillation,” J. Cardiovasc Electrophysiol, 1994, pp. 1045-1052, Vol. 5. At the beginning of a typical EP procedure, a catheter is inserted into a blood vessel near the groin of a patient and guided to the heart. The specialist will use specialized EP procedure tools to then conduct heart rhythm tests and, if warranted, treatment. Specifically, catheter ablation is a treatment that delivers/removes energy to create a discrete lesion of myocardial scar tissue (which may be in the form of a point, line or other shape and which is generally referred to herein as an ablation point). Catheter ablation may heat the heart tissue (e.g., radio frequency ablation (RFA)) or remove heat from the heart tissue (e.g., cryothermal energy ablation) to the point of causing lesions that will block certain electrical pathways in the heart tissue that are contributing to an arrhythmia. In ablation treatment of atrial fibrillation, specific sections of the pulmonary veins are primary ablation targets. Ablation treatment itself may be carried out using an irrigated ablation catheter.

Catheters are medical devices in the form of hollow flexible tubes for insertion into a part of the body usually to permit the passage of fluids or keep open a passageway. A catheter is normally accompanied with accessory components such as a control handle, catheter tips, surgical tools, etc., depending upon the application (and thus as a whole may be referred to, more properly, as a catheter system). In minimally invasive medical procedures, catheters are often used to deliver therapy in such a way that requires a respective catheter tip to be in contact with the tissue being treated. RFA is one example of such a procedure, wherein the therapy is carried out with an ablation catheter having a tip that delivers high frequency alternating current so as to cause heating of the tissue.

While some ablation procedures involve placing the ablation tip inside the tissue to be treated, such as in the treatment of tumors, others involve only touching the ablation tip directly against the tissue surface, such as in the treatment of cardiac arrhythmias. In the latter type of procedure, where the tip only touches the tissue surface without penetrating the tissue, the success of the procedure is partly dependent on how forcefully the ablation tip contacts the tissue surface. If the tip is not in good contact or in relatively light contact with the tissue surface, the heating therapy will be diminished. If the tip is firmly contacting the tissue surface with some force, the heating therapy will be more effective.

As noted above, in the case of a cardiac ablation procedure, the goal is to have the ablation catheter deliver energy to the heart tissue (or remove energy/heat) to the point of causing lesions that will block certain electrical pathways in the heart tissue that are contributing to the arrhythmia. Consequently, the degree of contact of the ablation tip against the tissue is highly important in the success of the therapy. To effectively block the electrical signal the lesions should have some depth within the tissue, as opposed to just being formed in a thin layer of the tissue surface. The depth of the lesion depends on, among other aspects (e.g., ablation time), both the contact force and the ablation power supplied to the tip. If lesions of sufficient depth and area are not being formed, because of insufficient contact and/or power, the ablation procedure will tend to be much longer and there will be a higher probability that the procedure will not be fully successful in stopping the arrhythmias. Conversely, if there is too much force and/or too much power, there are potential risks including penetration of the tissue wall, esophageal injury, cardiac tamponade or perforations from steam pops (particularly during irrigated ablation procedures at high power). Thus, successful cardiac ablation therapy seeks to form effective lesions while still minimizing the risk of complications. Both are dependent upon controlling the degree of contact of the ablation tip against the tissue.

Recent studies have emphasized the relevance of catheter tip-to-tissue contact force for quality or effectiveness of ablation points (this is described further in an article by V. Y. Reddy, entitled “Low catheter-tissue contact force results in late PV reconnection—initial results from EFFICAS I”, Heart Rhythm Society, 2011, and an article by D. C. Shah, V. Y. Reddy, J. Kautzner, N. Saoudi, C. H. Sikldy, P. Jais, G. Hindricks, A. Yulzari, H. Lambert, P. Neuzil, and K.-H. Kuck, entitled “Contact force during ablation predicts AF recurrence at 12 months,” Heart Rhythm Society, 2011). In these studies, the average contact force per patient was found to be correlated with the AFib recurrence rate. However, none of the previous studies explored if there is a relationship between the spatiotemporal force distribution and clinical outcome.

Such ablation and other EP procedures are routinely conducted under image guidance, for example, using mapping systems and/or X-ray fluoroscopy systems. The image guidance systems and techniques can provide live visualization of both a patient's anatomy and the catheter tip, and sometimes localization of the tip within some coordinate space, during a respective EP procedure. Since soft tissue resolution in X-ray images is very low, fluoroscopy systems may be used to superimpose additional information on the images, for example, a model of the anatomical structure, planned/targeted ablation locations, image information from pre-operative data, etc., for additional guidance. This is further described in an article by L. Zagorchev, R. Manzke, R. Cury, V. Reddy, and R. Chan, entitled “Rapid fusion of 2D x-ray fluoroscopy with 3D multislice CT for image-guided electrophysiology procedures”, Proceedings of SPIE, vol. 6509, 2007, p. 65092B.

However, although useful, such overlay image information offers only approximate guidance because of intra-operative heart beating motions, breathing motions, and catheter motions. While the medical professional has some feel of the resistance as a catheter is navigated towards the target anatomy via the image guidance, once at the target, there usually is not enough sensitivity for the medical professional to tell how good the contact is between the ablation tip and the tissue surface. Thus, by using imaging techniques alone, it can be very difficult to definitively judge whether or not an ablation tip is in good or appropriate contact with the tissue surface. To assist the medical professional, many catheter systems and methods measure tip contact force, usually relying on some form of sensor built into the tip, such as fiber optic force sensors, piezoelectric strain gauges or other such devices. Some catheter systems relay signals (electric, optical or fluid-based) back to the catheter's hand control, translating that signal into a corresponding force in an attempt to give a truer tactile feedback to the user. The TactiCath™ catheter (from Enclosense of Geneva, Switzerland) is an irrigated RF ablation catheter that provides contact force measurement. The TactiCath™ catheter uses a fiber-optic based force sensor that, from use in various studies and clinical trials, offers evidence of the clinical benefits of having a force sensing capability.

It would be advantageous to combine the force-sensing capabilities of catheters with the image guidance systems used in EP procedures in order to better relate catheter tip information to the actual location of the anatomy of interest. Such a combination could better provide and/or display information to the user to help gauge the force of the ablation tip contact and thus better control the degree of contact of the ablation tip against tissue. In that way, a cardiac ablation procedure, for example, may be accelerated while decreasing the risk of complications, like perforations. Importantly, there also can be a higher probability that the procedure will be fully successful in treating an arrhythmia.

### SUMMARY OF THE INVENTION

An embodiment of the invention obviates the above problems by providing an integrated system to support cardiac ablation procedures, comprising a) a catheter system having a catheter that is adapted to conduct ablation of heart tissue and a sensor that acquires measurements of contact force employed by the catheter in conducting ablations and b) an image guidance system that simultaneously provides a visualization of a cardiac ablation region and the acquired measurements and associated information of the contact force employed by the catheter in conducting a respective ablation of heart tissue. The sensor may comprise a fiber optic force sensor. The visualization may comprise an intra-procedural image of the cardiac ablation region and an overlay image. In such case, the overlay image may comprise either a pre-procedural image of the cardiac ablation region or a model of a corresponding cardiac ablation region.

The catheter system may provide contact force measurements for each conducted ablation to the image guidance system and the image guidance system may relate contact force measurements and associated information with a visualization location of a respective conducted ablation. In such case, the associated information may comprise contact force vectors obtained from contact force measurements or, alternatively, the associated information may comprise contact force-derived parameters obtained from contact force measurements. Also, the image guidance system may be adapted to manipulate the visualization of the cardiac ablation region, contact force measurements and associated information, and integration of the visualization and contact force measurements and associated information. Also, the image guidance system may further relate distribution of conducted ablations and contact force measurements and associated information with quality measures of the conducted ablations.

An embodiment of the invention may also provide a method of catheter navigation with contact force assessment to guide pulmonary vein isolation procedures, comprising: obtaining contact force values for each ablation lesion formed during an ablation of heart tissue for a respective pulmonary vein isolation procedure; associating each contact force value with the location of the respective ablation lesion in a visualization of a respective patient's heart and nearby vasculature; and providing the visualization integrated with the contact force values to guide the respective pulmonary vein isolation procedure. Each contact force value may comprise a force vector. Alternatively, each contact force value may comprise a contact force-derived parameter. The visualization of a respective patient's heart and nearby vasculature may comprise an intra-operative image of the heart and nearby vasculature and an overlay image. The overlay image may comprise either a pre-operative image of the respective patient's heart and nearby vasculature or a model of a heart and nearby vasculature. The visualization of a respective patient's heart and nearby vasculature may comprise locations of planned ablation lesions marked therein. The visualization integrated with the contact force values may comprise relating the spatiotemporal distribution of contact force values with quality measures of the contact force values.

An embodiment of the invention may also provide a system of providing image guidance for medical procedures, comprising: an imager that acquires fluoroscopic images and other image data of an anatomical structure of a patient and a processor that manipulates the fluoroscopic images and other image data and catheter tip-to-tissue contact force measurements taken during a catheterization procedure to produce an integrated visualization of the catheterization procedure and of effectiveness assessments of the catheter tip-to-tissue contact forces during the procedure. The catheterization procedure may comprise a pulmonary vein isolation procedure. The integrated visualization may comprise an integrated image having a spatiotemporal distribution of a contact force-derived parameter with quality measures of the respective or other contact force-derived parameter. The processor may produce a real-time or near real-time integrated visualization.

### DESCRIPTION OF THE DRAWINGS

For a better understanding of the present invention, reference is made to the following description of exemplary embodiments thereof, and to the accompanying drawings, wherein:

FIG. 1 is a block diagram of a catheter navigation system constructed in accordance with an embodiment of the present invention;

FIG. 2 is a flow chart of a catheter navigation method carried out in accordance with an embodiment of the present invention;

FIG. 3 is a model representation of an ablation catheter inserted into the heart of a respective patient;

FIG. 4 shows a display of anatomical imaging with contact force information for performing an ablation procedure;

FIG. 5 shows a second display of anatomical imaging with contact force information for performing an ablation procedure;

FIG. 6 shows a post-procedural display of contact force information of a previously-performed ablation procedure; and

FIG. 7 shows a second post-procedural display of contact force information of a previously-performed ablation procedure.

### DETAILED DESCRIPTION

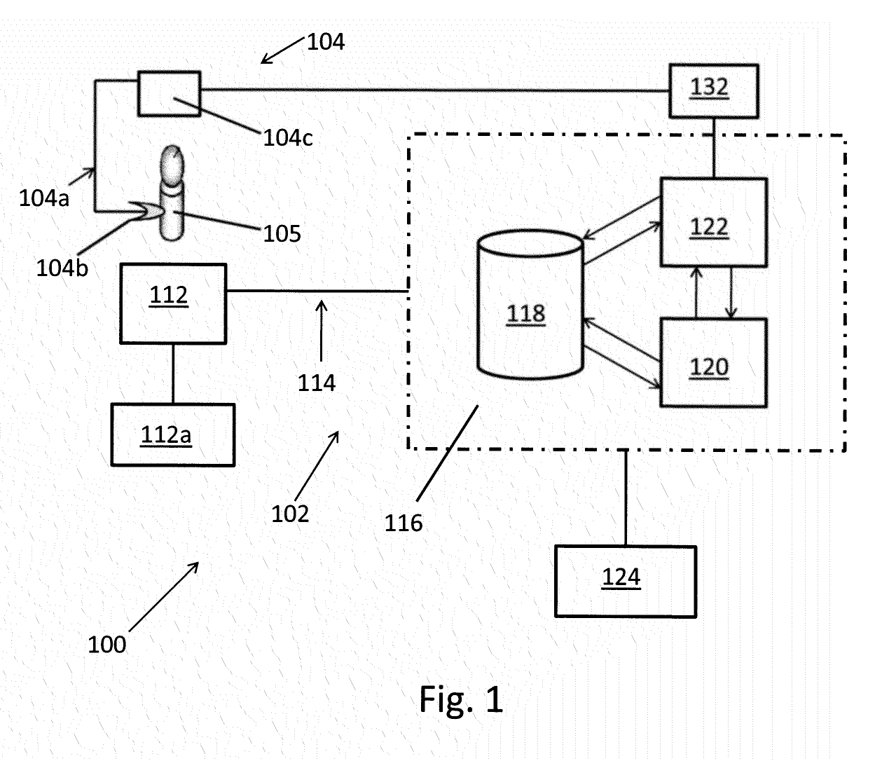

FIG. 1 is a block diagram of a catheter navigation system 100 constructed in accordance with an embodiment of the present invention. The navigation system 100 comprises an image guidance system 102 that implements fluoroscopy-based catheter navigation assistance (e.g., a biplane C-arm fluoroscopy system) and a catheter system 104. The image guidance system 102 comprises a medical imaging scanner 112 that acquires image data of a patient 105 under examination and more specifically of a region of interest, for example, the heart of the patient 105. As noted above, the scanner 112 may use X-ray imaging or other appropriate imaging modality to acquire the image data, such as, fluoroscopy sequences, 3D datasets (C-arm CT imaging), and 2D DSA sequences. The scanner 112 may acquire raw image data from multiple scanned views of the region of interest of the patient 105, record or reconstruct the images, and produce image data signals for the multiple views. This may be done in real-time or near real-time. The image data signals may be in Digital Imaging and Communications in Medicine (DICOM) format. Other formats may also be used.

The imaging scanner 112 is operably connected to a computer system 112a that controls the operation of the scanner 112 and, via a communication channel 114, to an image processing system 116 that processes the image data signals utilizing appropriate image processing software applications. The image processing system 116 has an image data archive or database 118, an application server 120, and a user workstation 122. The components of the image processing system 116 are interconnected via a communications network that may be implemented by physical connections, wireless communications, or a combination. The image data archive or database 118 is adapted to store the image data signals that are produced by the imaging scanner 112 as well as the results of any additional operations on the image data signals by the other components of the image processing system 116. The image data archive or database 118 is also adapted to store pre-acquired imaging data (obtained via any appropriate imaging modality) or models of the anatomy or region of interest as well as other externally-generated data. The image data archive or database 118 may be a Picture Archiving and Communications System (PACS). Other types of image data archives or databases may also be used.

The user workstation 122 is adapted to control the operation of the imaging processing system 116 and its various components. The user workstation 122 particularly operates the application server 120 and the various image processing software applications that are stored in, or are accessible by, the server 120. The application server 120 also manages and coordinates the image data sets among the image processing applications. The image processing applications may include, for example, visualization applications, computer-aided diagnosis (CAD) applications, medical image rendering applications, anatomical segmentation applications, image registration applications, or any other type of medical image processing application. The image processing applications may also include methods that are carried out in accordance with embodiments of the present invention and those of the respective various steps. The image data archive or database 118, applications server 120, and the user workstation 122 may also each be connected to a remote computer network 124 for communication purposes or to access additional data or functionality. The workstation 122 may comprise appropriate user interfaces, like displays, storage media, input/output devices, etc.

The catheter system 104 comprises an ablation catheter 104a having a catheter tip 104b with an integrated force sensor that gauges or measures the contact force of the tip 104b against a tissue. The forcer sensor may take on various forms, such as, a fiber optic force sensor, piezoelectric strain gauge, or other such devices. The catheter system 104 also has an interface 104c that allows force information (e.g., force amplitudes, force directions, etc.) picked up by the force sensor to be shared with the image guidance system 102 and other systems used by the medical professional for a respective medical procedure, e.g., other mapping systems or recording systems. The catheter system interface 104c may provide an analog output with two channels that can send the current force information, as well as force-derived parameters (for example, the force-time integral) if so adapted. As seen in the figure, the catheter system interface 104c may utilize a conventional A/D converter 132 to digitize the output analog signal and to connect the interface 104c to the image processing system 116 of the image guidance system 102, via, for example, a USB connection to the user workstation 122. The user workstation 122 may then operate to manipulate the catheter system interface 104c output data as desired and transfer it to an appropriate user interface connected thereto, for example, displaying the interface 104c output data concurrently with the anatomical imaging in a specified format, either on the same monitor or on a separate monitor, in the operating room for the respective procedure. The user workstation 122 may manipulate the catheter system interface 104c output data to obtain force-derived parameters (for example, the force-time integral) in various formats. Note that the A/D converter 132 may or may not be part of the catheter system interface 104c.

Such a catheter system 104 may be provided by the above-mentioned TactiCath™ catheter system. The force-time integral is further described in an article by D. Shah, H. Lambert, H. Nakagawa, A. Langenkamp, N. Aeby, and G. Leo, entitled “Area under the real-time contact force curve (force-time integral) predicts radiofrequency lesion size in an in vitro contractile model”, Journal of Cardiovascular Electrophysiology, Vol. 21, No. 9, pp. 1038-1043, 2010.

The various components of the image guidance system 102 are well known components. They may be configured and interconnected in various ways as necessary or as desired. The image guidance system 102 and, in particular, the image processing system 116 is adapted to permit the image guidance system 102 to operate and to implement methods in accordance with embodiments of the invention, for example, as shown and described below. Advantageously, the catheter system interface 104c output data may be integrated with an image guidance system, such as biplane C-arm fluoroscopy system, that enables localization and reconstruction of 3-D points, e.g., the catheter tip 104b, from two 2-D X-ray views (as further described in an article by A. Brost, N. Strobel, L. Yatziv, W. Gilson, B. Meyer, J. Hornegger, J. Lewin, and F. Wacker, entitled “Geometric Accuracy of 3-D X-Ray Image-Based Localization from Two C-Arm Views”, Workshop on Geometric Accuracy In Image Guided Interventions-Medical Image Computing and Computer Assisted Interventions, MICCAI, 2009, pp. 12-19 and which is hereby incorporated by reference).

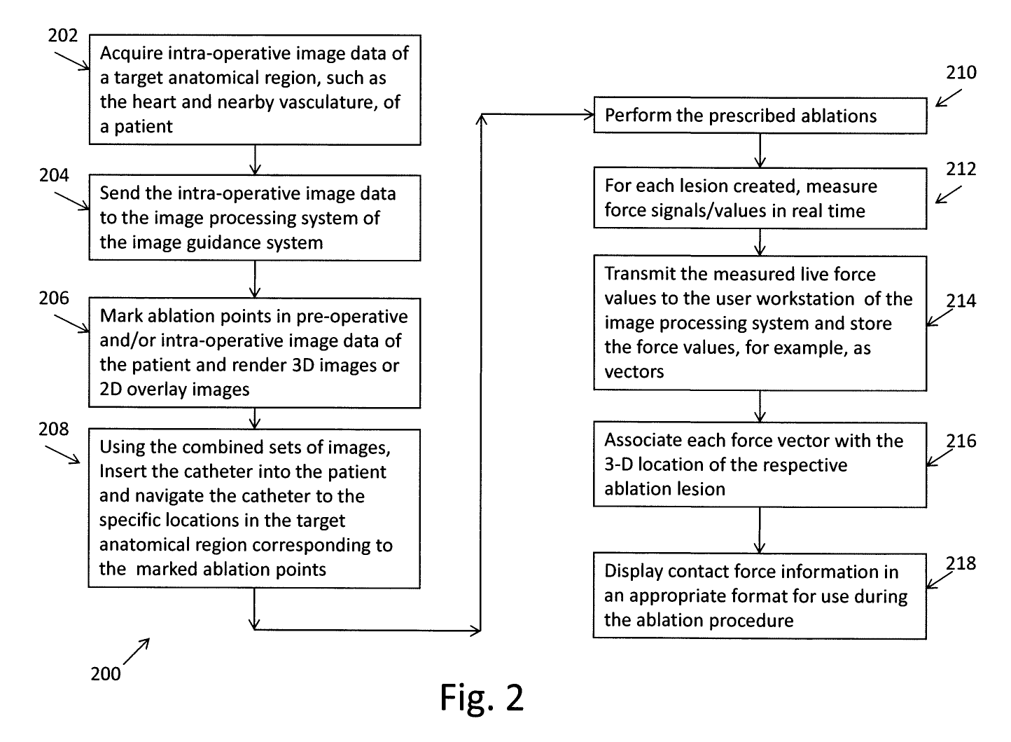

FIG. 2 is a flow chart of a catheter navigation method 200 (simplified) carried out in accordance with an embodiment of the present invention. The catheter navigation system 100 may utilize the method 200 of FIG. 1. During a respective medical procedure, like cardiac ablation, a medical professional operates the medical imaging scanner 112 of the image guidance system 102 that acquires intra-operative image data of a target anatomical region, such as the heart and nearby vasculature, of the patient 105 under examination (Step 202). As noted above, the image guidance system 102 may be a biplane C-arm fluoroscopy system. The intra-operative images and associated data conveying projection geometry parameters of the projection system are sent to the image processing system 116 of the image guidance system 102 (Step 204). Using the user workstation 122, a medical professional operates imaging applications that assist in marking specific planned/targeted ablation points and other landmarks, in pre-operative and intra-operative image data of the patient 105. Markings or annotations may take on various forms, for example, different colorations of objects, different formatting of objects, etc. This information may be rendered either in three dimension (3-D) images or as two-dimensional (2-D) fluoroscopic overlay images, for example, that can be overlaid the intra-operative images (Step 206). Further, the medical professional operates imaging applications that assist in displaying the imaging data and associated data and the overlay imaging data.

A medical professional uses the displayed information to properly situate the ablation catheter 104a in the patient 105, i.e., insert the catheter 104a into the patient 105 and navigate the ablation catheter 104a to the target anatomical region and specific locations corresponding to the previously-marked ablation points (Step 208). After the catheter insertion, a medical professional may again (or for the first time) use the user workstation 122 to mark specific planned ablation points and/or points of already-ablated tissue (i.e., conducted ablation points), and other landmarks, in pre-operative and intra-operative image data of the patient 105. This information may be rendered either in three dimension (3-D) images or as two-dimensional (2-D) fluoroscopic overlay images, for example, that can be overlaid the intra-operative images.

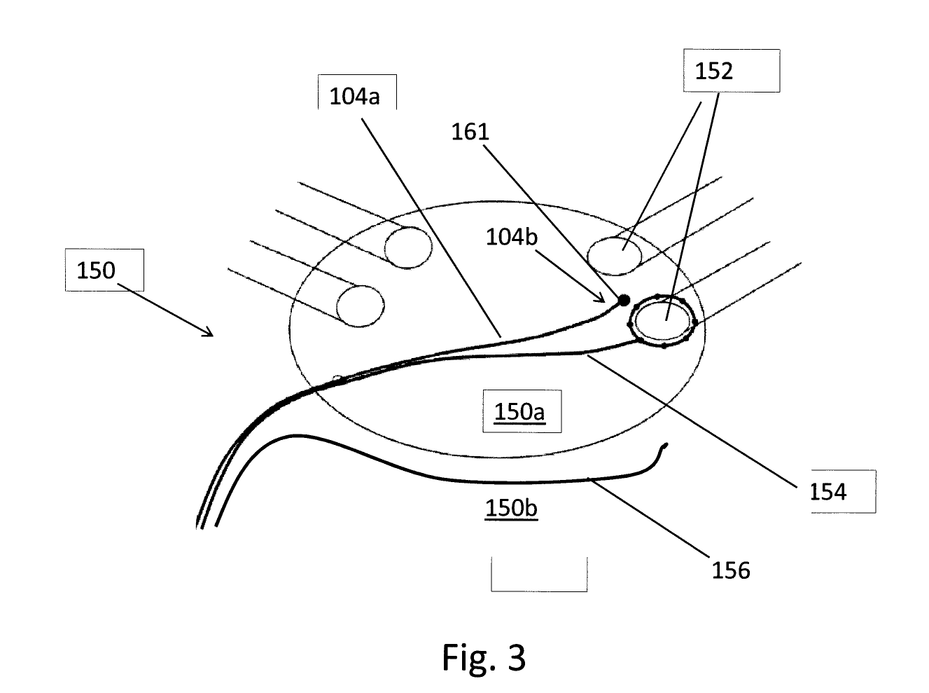

FIG. 3 is a model representation of the ablation catheter 104a inserted into the heart 150 of a respective patient 105. The catheter tip 104b (with the integrated force sensor) is situated at an ablation point 161 in the left atrium 150a of the heart 150. To provide a frame of reference, the pulmonary vein ostia 152 nearby the catheter tip 104b are shown in the figure as two circles. The figure also shows a mapping catheter 154 which is a diagnostic catheter used with the ablation catheter 104a for mapping electrical conduction between the left atrium 150a and the pulmonary veins. The figure also shows a coronary sinus (CS) catheter 156 which is a diagnostic catheter used with the respective ablation catheter 104a that is placed at the coronary sinus 150b lying between the left atrium 150a and left ventricle for visualizing the activation of both the left atrium 150a and left ventricle.

Once the catheter 104a is situated, a medical professional operates the catheter system 104 to perform the prescribed (i.e., targeted or planned) ablations (Step 210). For each lesion created by the medical professional, the catheter system 104 measures a force signal/value (e.g., amplitude and direction) at the catheter tip 104b in real time (Step 212). The catheter system interface 104c transmits the measured live force values, via the A/D converter 132, to the user workstation 122 of the image processing system 116 which may store the force values, for example, as vectors (Step 214) for further processing and/or visualization. The image data archive or database 118 may also be used for storage. Regardless, the user workstation 122 may transmit the stored values to the application server 120 as appropriate. Using the user workstation 122, a medical professional operates imaging applications that assist in evaluating each force vector individually. For example, due to the 3-D catheter localization capabilities of the image guidance system 102, the image processing system 116 can associate a force vector with the 3-D location of the respective ablation lesion (Step 216). Further, the image processing system 116 may compute force-time plots, force-time integral (FTI), or other force-derived parameters. The contact force information, including the measured live force values, can then be displayed, in an appropriate format and on a specified user interface, for use during the respective ablation procedure (Step 218).

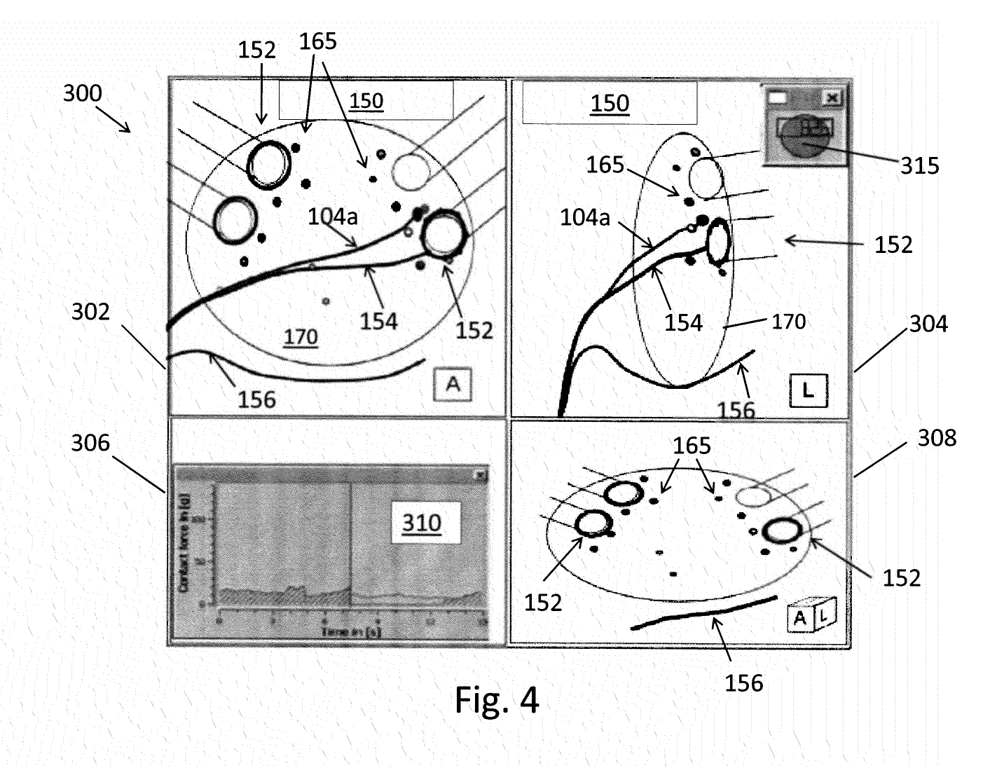

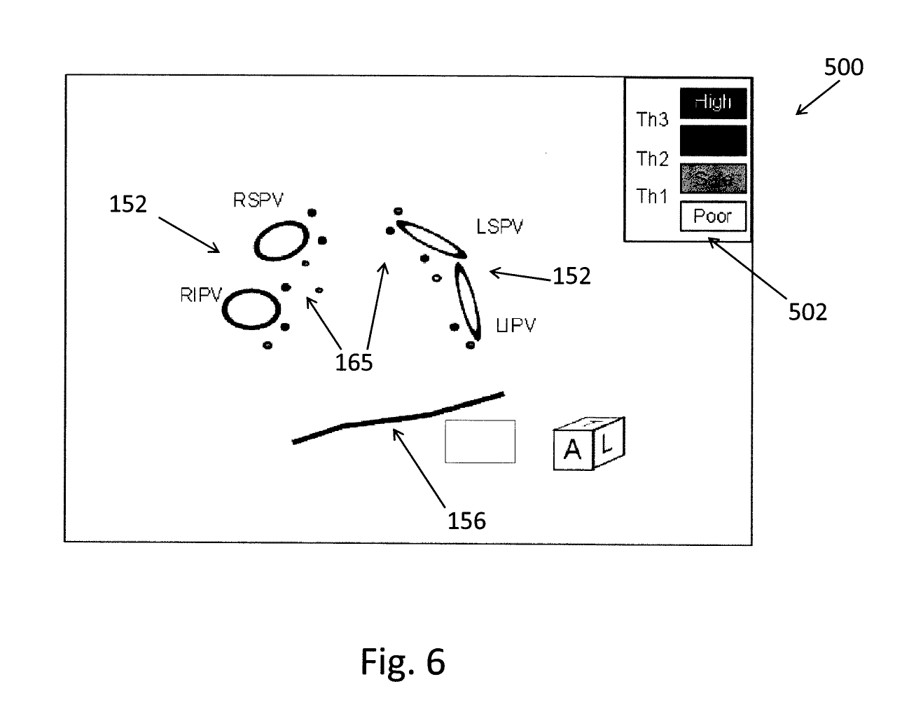

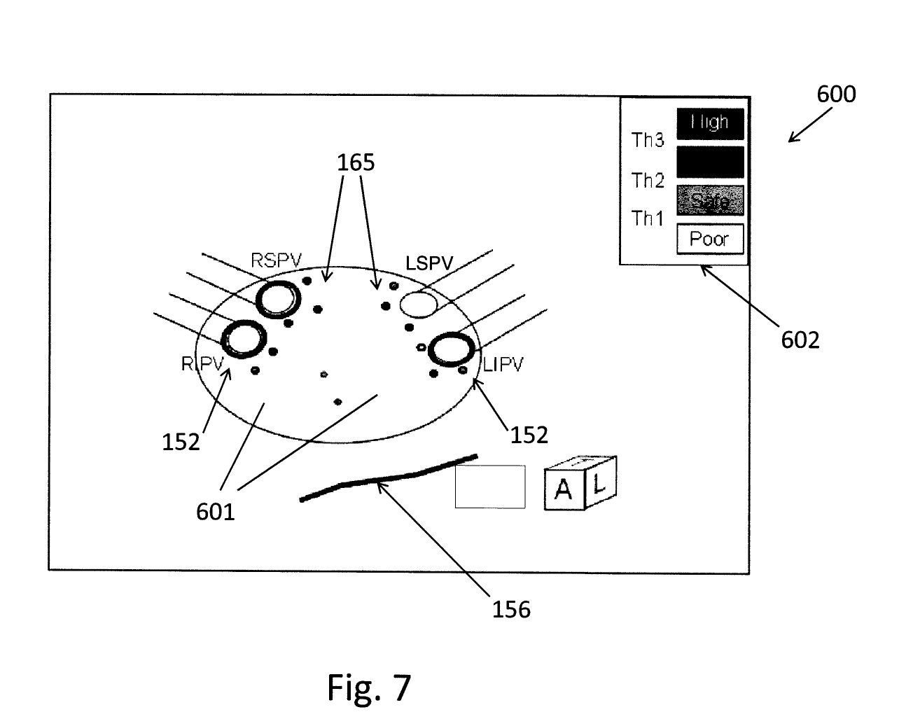

FIG. 4 illustrates an exemplary display 300 on a user interface. The display 300 combines anatomical imaging with force vector information to assist a medical professional in performing a respective ablation procedure. The display 300 is formatted to have four sections or quadrants 302, 304, 306, 308 of information. The top two quadrants 302, 304 contain individually oriented fluoroscopy images, e.g., front (anterior) and lateral, respectively, with landmarks annotated, of an ablation catheter 104a inserted into the heart 150 of a respective patient 105. The top two quadrants 302, 304 show actual fluoroscopy images of the heart 150 in the background (not shown for ease of visualization) and may also contain an overlay image 170 of a three-dimensional left atrium model. The two top quadrants 302, 304 specifically show the ablation catheter 104a, the mapping catheter 154, and the CS catheter 156 as marked (represented in the figure as bolded elements). Three pulmonary vein ostia 152 and the ablation points 165 have also been marked. The lower left quadrant 306 contains a visualization as a force-time plot 310 of the contact force of the ablation catheter 104a being used in an ablation procedure shown in the top two quadrants 302, 304. As noted above, typically, the image processing system 116 computes both the force-time plot and the FTI (or other force-derived parameters) based on the measured live contact signals received from the catheter system 104. Force is scaled in units of grams and time is scaled in units of seconds, although other scales may be used. As will be further described with respect to FIGS. 6 and 7, the lower right quadrant 308 contains a 3-D visualization of the distribution of conducted ablation points 165, each of which having a color (or other feature) assigned based on FTI value or other force-derived parameters (e.g. ablation duration, average contact force). The visualization may be set against a neutral background (with or without the overlay image 170) and shows annotations of landmarks, including for example the conducted ablation points 165, the CS catheter 156, and three pulmonary vein ostia 152. Other landmarks not shown may also be annotated e.g., the coronary sinus, as desired.

As noted above, the various markings/annotations in the display 300 may be distinguished, for example, using different colorizations of the objects. Also, during a respective ablation procedure, the images may be further marked as desired to distinguish planned ablation points from points of already-ablated tissue, i.e., conducted ablation points. As noted above, the markings/annotations and the overlay 170 provide additional guidance for the live visualization of the patient's anatomy and the catheter tip 104b during a respective ablation procedure.

In the top right corner of the top right quadrant 304, the display 300 presents an icon 315 that is a color-coded threshold classifier for the force-time integral (FTI) for the current ablation. Alternatively, other force-derived parameters that influence the ablation lesion could be shown instead. The icon 315 may be colored using a scale that indicates the value of the FTI for the current ablation being performed. Other types of coding may also be used, for example, texture-coding, in place of the color-coding. Also, other shapes and forms for the icon 315 may be used.

Note that different fluoroscopy orientation settings for the images are common, e.g., 0 and 90 degrees (front, lateral) or −30 and 60 degrees, etc. The figure denotes the fluoroscopy orientation views for the top two quadrants 302, 304 and the lower right quadrant 308 via the use of a legend in the lower right hand corner of each quadrant (i.e., A in a square for anterior view, L in a square for lateral view, and A and L in a cube for a perspective or 3D-like view). This legend or an equivalent may or may not be included as part of a display 300.

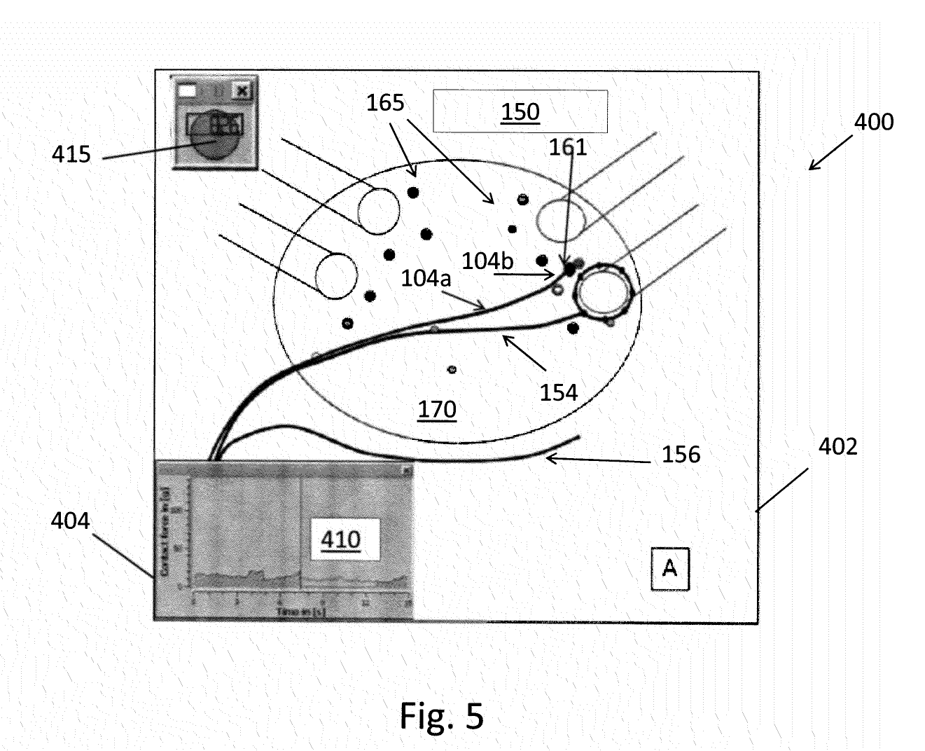

FIG. 5 illustrates a second exemplary display 400 on a user interface. The display 400 also combines anatomical imaging with force vector information during a respective ablation procedure. The display 400 is formatted to have two windows or sections 402, 404 of information. The larger window 402 contains a fluoroscopy image similar to the front (anterior) fluoroscopy image shown in FIG. 4, with landmarks annotated, of an ablation catheter 104a inserted into the heart 150 of a respective patient 105. The larger window 402 shows an actual fluoroscopy image of the heart 150 in the background (not shown for ease of visualization) and may also contain an overlay image 170 of a three-dimensional left atrium model. The catheter tip 104b is situated at an ablation point 161 (which has been marked). The ablation catheter 104a, the mapping catheter 154, the CS catheter 156, and ablation points 165 are also marked (represented in the figure as bolded elements). The smaller window 404 contains a visualization as a force-time plot 410 of the contact force of the ablation catheter 104a being used in an ablation procedure shown in the larger window 402. As noted above, typically, the image processing system 116 computes both the force-time plot and the FTI (or other force-derived parameters) based on the measured live contact signals received from the catheter system 104. Force is scaled in units of grams and time is scaled in units of seconds, although other scales may be used. At the top left corner of the larger window 402, the display 400 presents an icon 415 that is a color-coded threshold classifier for the force-time integral (FTI) for the current ablation being performed. As noted above, other force-derived parameters that influence the ablation lesion could be shown instead. The icon 415 may be colored using a scale that indicates the value of the FTI for the current ablation being performed. Also as noted above, other types of coding and other types of icons 415 may be used.

FIG. 6 illustrates a third exemplary display 500 on a user interface. The display 500 provides a post-procedural visualization of contact force information of a previously-performed ablation procedure. The visualization is similar to the visualization provided in the lower right quadrant 308 of the display 300 shown in FIG. 4. The display 500 shows a distribution of the conducted ablation points 165, each of which having a color (or other feature) assigned based on FTI value or other force-derived parameter (e.g. ablation duration, average contact force). The FTI-color scale 502 in the right corner forms part of the display 500. Note that the scale 502 provides both quantitative (high, normal and low) and qualitative (high, safe and poor) information to indicate the overall quality of a contact force value and thus each conducted ablation point 165. Although the conducted ablation points 165 are shown against a neutral background, the distribution of the conducted ablation points 165 corresponds to the actual anatomical locations of the points 165. The visualization shows annotations of landmarks, including for example the conducted ablation points 165, the CS catheter 156, and pulmonary vein ostia 152. The pulmonary vein ostia 152 are labeled in the figure to provide a frame of reference, RSPV (right superior pulmonary vein), RIPV (right inferior pulmonary vein), LSPV (left superior pulmonary vein), and LIPV (left inferior pulmonary vein); however, the labeling may or may not be part of the display 500. Also, the fluoroscopy view legend shown in the lower right corner of the figure may or may not be part of the display 500. The visualization may be adapted to include an overlay image 170 (not shown).

FIG. 7 illustrates a fourth exemplary display 600 on a user interface. The display 600 provides a post-procedural visualization of contact force information of a previously-performed ablation procedure. The display 600 shows a distribution of the conducted ablation points 165 within a 3D anatomical context. Specifically, the conducted ablation points 165 are shown against a 3D background 601 of a left atrium extracted from a pre-operative whole heart CT data set. Each of the conducted ablation points 165 has a color (or other feature) assigned based on FTI value or other force-derived parameter (e.g. ablation duration, average contact force). The FTI-color scale 602 in the right corner forms part of the display 600. The scale 602 provides both quantitative (high, normal and low) and qualitative (high, safe and poor) information to indicate the overall quality of a contact force value and thus each conducted ablation point 165. The visualization shows annotations of landmarks, including for example the conducted ablation points 165, the CS catheter 156, and three pulmonary vein ostia 152. Like FIG. 6, the pulmonary vein ostia 152 are labeled to provide a frame of reference, RSPV (right superior pulmonary vein), RIPV (right inferior pulmonary vein), LSPV (left superior pulmonary vein), and LIPV (left inferior pulmonary vein); however, the labeling may or may not be part of the display 600. Also, the fluoroscopy view legend shown in the lower right corner of the figure may or may not be part of the display 600.

Advantageously, the integrated catheter navigation system 100 allows a detailed evaluation of catheter tip-to-tissue contact during the ablation procedure (i.e., quality or effectiveness assessments) taking into account the 3-D position of each lesion created. Also, the integrated catheter navigation system 100 provides live visualization of the ablation catheter 104a contact force on the fluoroscopy images as well as extended evaluation (intra-operative and post-operative) possibilities about contact force applied during the ablation procedure. The integrated catheter navigation system 100 permits a determination of which anatomical locations the contact force is applied as a well as of how the contact force is distributed throughout a ablation procedure.

Other modifications are possible within the scope of the invention. For example, the patient 105 may be an animal subject or any other suitable object rather than a human patient. Also, the invention may used in other catheter-based procedures and for other anatomical regions of interest. Also, the ablation catheter 104a may perform ablation differently than as described and may perform other operations. Also, the catheter system 104 may provide digital output signals instead of analog signals. In such case, a digital interface between the catheter system 104 and the image processing system 116 may be utilized (without the need for an A/D converter 132). Also, the catheter system 104, instead of the image processing system 116, may be adapted to compute the FTI (or other force-derived parameters) based on the measured live contact signals. Also, the biplane C-arm fluoroscopy system may be, for example, the Siemens Ards zee Biplane system. Also, the A/D converter 132 may be a device from Pico Technology (St Neats, Cambridgeshire, United Kingdom).

In addition, although the steps of the catheter navigation method 200 has been described in a specific sequence, the order of the steps may be re-ordered in part or in whole and the steps may be modified, supplemented, or omitted as appropriate. Also, the method 200 may use various well known algorithms and software applications to implement the steps and substeps. Further, the method 200 may be implemented in a variety of algorithms and software applications. Further, the method 200 may be supplemented by additional steps or techniques. It is also understood that the method 200 may carry out all or any of the steps using real-time data, stored data from a data archive or database, data from a remote computer network, or a mix of data sources.

Also, the various described instrumentation and tools may be configured and interconnected in various ways as necessary or as desired. Further, although in the described method 200 the user may use self-contained instrumentation and tools, the user may use other instrumentation or tools in combination with or in place of the instrumentation and tools described for any step or all the steps of the methods 200, including those that may be made available via telecommunication means. Further, the described method 200, or any steps, may be carried out automatically by appropriate instrumentation and tools or with some manual intervention.

What is claimed is:

1. An integrated system to support cardiac ablation procedures, comprising a) a catheter system having a catheter that is adapted to conduct ablation of heart tissue and a sensor that acquires measurements of contact force employed by the catheter in conducting ablations and b) an image guidance system that simultaneously provides a visualization of a cardiac ablation region and the acquired measurements and associated information of the contact force employed by the catheter in conducting a respective ablation of heart tissue.

2. The system of claim 1, wherein the sensor comprises a fiber optic force sensor.

3. The system of claim 1, wherein the visualization comprises an intra-procedural image of the cardiac ablation region and an overlay image.

4. The system of claim 3, wherein the overlay image comprises either a pre-procedural image of the cardiac ablation region or a model of a corresponding cardiac ablation region.

5. The system of claim 1, wherein the catheter system provides contact force measurements for each conducted ablation to the image guidance system and the image guidance system relates contact force measurements and associated information with a visualization location of a respective conducted ablation.

6. The system of claim 5, wherein the associated information comprises contact force vectors obtained from contact force measurements.

7. The system of claim 5, wherein the associated information comprises contact force-derived parameters obtained from contact force measurements.

8. The system of claim 5, wherein the image guidance system is adapted to manipulate the visualization of the cardiac ablation region, contact force measurements and associated information, and integration of the visualization and contact force measurements and associated information.

9. The method of claim 5, wherein the image guidance system further relates distribution of conducted ablations and contact force measurements and associated information with quality measures of the conducted ablations.

10. A method of catheter navigation with contact force assessment to guide pulmonary vein isolation procedures, comprising:
a. obtaining contact force values for each ablation lesion formed during an ablation of heart tissue for a respective pulmonary vein isolation procedure;
b. associating each contact force value with the location of the respective ablation lesion in a visualization of a respective patient's heart and nearby vasculature; and
c. providing the visualization integrated with the contact force values to guide the respective pulmonary vein isolation procedure.

11. The method of claim 10, wherein each contact force value comprises a force vector.

12. The method of claim 10, wherein each contact force value comprises a contact force-derived parameter.

13. The method of claim 10, wherein the visualization of a respective patient's heart and nearby vasculature comprises an intra-operative image of the heart and nearby vasculature and an overlay image.

14. The method of claim 13, wherein the overlay image comprises either a pre-operative image of the respective patient's heart and nearby vasculature or a model of a heart and nearby vasculature.

15. The method of claim 10, wherein the visualization of a respective patient's heart and nearby vasculature comprises locations of planned ablation lesions marked therein.

16. The method of claim 10, wherein providing the visualization integrated with the contact force values comprises relating the spatiotemporal distribution of contact force values with quality measures of the contact force values.

17. A system of providing image guidance for medical procedures, comprising: an imager that acquires fluoroscopic images and other image data of an anatomical structure of a patient and a processor that manipulates the fluoroscopic images and other image data and catheter tip-to-tissue contact force measurements taken during a catheterization procedure to produce an integrated visualization of the catheterization procedure and of effectiveness assessments of the catheter tip-to-tissue contact forces during the procedure.

18. The system of claim 17, wherein the catheterization procedure comprises a pulmonary vein isolation procedure.

19. The system of claim 17, wherein the integrated visualization comprises an integrated image having a spatiotemporal distribution of a contact force-derived parameter with quality measures of the respective or other contact force-derived parameter.

20. The system of claim 17, wherein the processor produces a real-time or near real-time integrated visualization.
